(19) United States
(12) Patent Application Publication
(10) Pub. No.: US 2022/0379003 A1
(43) Pub. Date: Dec. 1, 2022

(54) DRESSING WITH AREA MANAGEMENT FOR EXTREMITIES

(71) Applicant: KCI Licensing, Inc., San Antonio, TX (US)

(72) Inventors: Jonathan G. REHBEIN, San Antonio, TX (US); Larry Tab RANDOLPH, San Antonio, TX (US); Richard Marvin KAZALA, Jr., San Antonio, TX (US)

(73) Assignees: KCI Licensing, Inc., San Antonio, TX (US); KCI Licensing, Inc., San Antonio, TX (US)

(21) Appl. No.: 17/772,479

(22) PCT Filed: Oct. 29, 2020
(86) PCT No.: PCT/IB2020/060158

§ 371 Date: Apr. 27, 2022

Related U.S. Application Data
(60) Provisional application No. 62/955,534, filed on Dec. 31, 2019.
(60) Provisional application No. 62/929,215, filed on Nov. 1, 2019.

(51) Int. Cl. A61M 1/00; A61F 13/06

(52) U.S. Cl. CPC A61M 1/917; A61F 13/06; A61M 1/915; A61F 13/10

(57) ABSTRACT

A manifold for use with a dressing for treating an area around an extremity of a patient may include a concave recess positioned at a first end of the manifold. A fold axis may bisect the concave recess and extend from the first end of the manifold to a second end of the manifold. A first portion of the manifold may extend orthogonal to the fold axis toward a first side of the manifold, and a second portion of the manifold may extend orthogonal to the fold axis toward a second side of the manifold opposite to the first side. The fold axis may be configured to extend lengthwise along the extremity. Additionally disclosed are other apparatus, dressings, systems, and methods.

### CROSS-REFERENCE TO RELATED APPLICATIONS

This application claims the benefit of priority to U.S. Provisional Application No. 62/929,215, filed on Nov. 1, 2019 and U.S. Provisional Application No. 62/955,534, filed on Dec. 31, 2019, both of which are incorporated herein by reference in their entirety.

### TECHNICAL FIELD

This disclosure relates generally to tissue treatment systems and more particularly, but without limitation, to tissue area management of one or more extremities of a patient using reduced-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for managing tissue sites in a negative-pressure therapy environment are set forth in the appended claims. The following description provides non-limiting, illustrative example embodiments to enable a person skilled in the art to make and use the claimed subject matter.

In some example embodiments, a dressing for treating an area around an extremity with reduced pressure may include an attachment device, a manifold, and a cover. The attachment device may include a treatment aperture. The manifold may be configured to be at least partially exposed to the area around the extremity through the treatment aperture. The manifold may include a concave recess, a fold axis, a first conformable area, and a second conformable area. The concave recess may extend into an edge of the manifold at a first end of the manifold. The fold axis may bisect the concave recess and extend from the first end to a second end of the manifold along a length of the manifold. The first conformable area may extend orthogonal to the fold axis along a width of the manifold and toward a first side of the manifold. The second conformable area may extend orthogonal to the fold axis along the width of the manifold and toward a second side of the manifold opposite to the first side. At least a portion of the width of the manifold may increase from the first end to the second end. The cover may be configured to be disposed over the manifold and coupled to the attachment device around the manifold.

In some example embodiments, a method for treating an area around an extremity with reduced pressure may include applying a dressing including a fold axis to an extremity such that the fold axis extends lengthwise along the extremity. Further, the method may include wrapping a first conformable area of the dressing circumferentially around the extremity in a first direction, and wrapping a second conformable area of the dressing circumferentially around the extremity in a second direction opposite to the first direction. Further, the method may include fluidly coupling a reduced-pressure source to a manifold of the dressing and delivering reduced pressure from the reduced-pressure source to the manifold.

In some example embodiments, a manifold for use with a dressing for treating an area around an extremity with reduced pressure may include a concave recess, a fold axis, a first conformable area, and a second conformable area. The concave recess may extend into an edge of the manifold at a first end of the manifold. The fold axis may bisect the concave recess and extend from the first end of the manifold to a second end of the manifold along a length of the manifold. The first conformable area may extend orthogonal to the fold axis along a width of the manifold and toward a first side of the manifold. The second conformable area may extend orthogonal to the fold axis along the width of the manifold and toward a second side of the manifold opposite to the first side. The first conformable area may be symmetrical to the second conformable area across the fold axis.

In some example embodiments, a manifold for use with a dressing for treating an area around an extremity with reduced pressure may include a concave recess, a fold axis, a first portion, and a second portion. The concave recess may be positioned at a first end of the manifold. The fold axis may bisect the concave recess and extend from the first end of the manifold to a second end of the manifold. The first portion of the manifold may extend orthogonal to the fold axis toward a first side of the manifold. The second portion of the manifold may extend orthogonal to the fold axis toward a second side of the manifold opposite to the first side. The fold axis may be configured to extend lengthwise along the extremity.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system that can provide reduced-pressure therapy in accordance with this specification;

FIG. 2 is a graph illustrating example pressure control modes that may be associated with some example embodiments of the therapy system of FIG. 1;

FIG. 3 is a graph illustrating another example pressure control mode suitable for some example embodiments of the therapy system of FIG. 1;

FIG. 4 is a top plan view of an example embodiment of a dressing that may be associated with an example embodiment of the therapy system of FIG. 1;

FIG. 5 is an exploded, perspective view of the dressing of FIG. 4, illustrating additional details that may be associated with some examples;

FIG. 6 is a side cut-away view of the example dressing of FIG. 4 in an assembled state, taken at line 6-6 in FIG. 4, illustrating additional details that may be associated with some examples;

FIG. 7A illustrates an example step of application of the example dressing of FIG. 4 to a leg of a patient;

FIG. 7B illustrates an example subsequent or final step of application of the example dressing shown in FIG. 7A;

FIG. 8 illustrates the example dressing of FIG. 4 applied to an arm of a patient;

FIG. 9 is a top plan view of another example embodiment of a dressing that may be associated with an example embodiment of the therapy system of FIG. 1;

FIG. 10 is a top plan view of yet another example embodiment of a dressing that may be associated with an example embodiment of the therapy system of FIG. 1; and

FIG. 11 is a top plan view of yet another example embodiment of a dressing that may be associated with an example embodiment of the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and non-limiting.

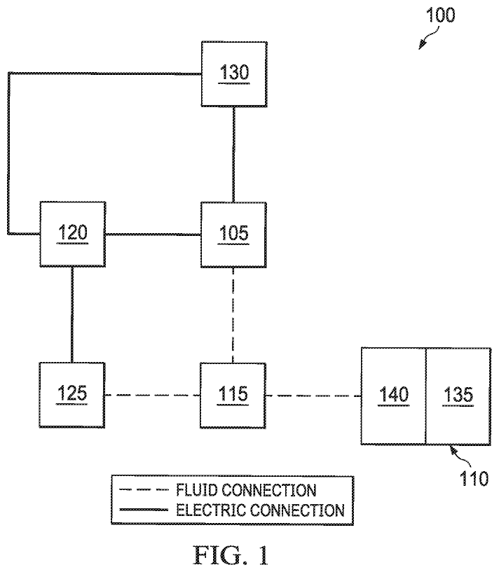

FIG. 1 is a block diagram of an example embodiment of a therapy system 100 that can provide reduced-pressure therapy to a tissue site in accordance with this specification. The term “tissue site” in this context may refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, grafts, and incisions, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of reduced pressure, such as a reduced-pressure source 105, a dressing 110, a fluid container, such as a container 115, and a regulator or controller, such as a controller 120, for example. Additionally, the therapy system 100 may include sensors to measure operating parameters and provide feedback signals to the controller 120 indicative of the operating parameters. As illustrated in FIG. 1, for example, the therapy system 100 may include one or more sensors coupled to the controller 120, such as a first sensor 125 and a second sensor 130.

As illustrated in the example of FIG. 1, the dressing 110 may include a tissue interface 135, a cover 140, or both in some embodiments.

Some components of the therapy system 100 may be housed within or used in conjunction with other components, such as sensors, processing units, alarm indicators, memory, databases, software, display devices, or user interfaces that further facilitate therapy. For example, in some embodiments, the reduced-pressure source 105 may be combined with the controller 120 and other components into a therapy unit.

In general, components of the therapy system 100 may be coupled directly or indirectly. For example, the reduced-pressure source 105 may be directly coupled to the container 115, and may be indirectly coupled to the dressing 110 through the container 115. Coupling may include fluid, mechanical, thermal, electrical, or chemical coupling (such as a chemical bond), or some combination of coupling in some contexts. For example, the reduced-pressure source 105 may be electrically coupled to the controller 120, and may be fluidly coupled to one or more distribution components to provide a fluid path to a tissue site. In some embodiments, components may also be coupled by virtue of physical proximity, being integral to a single structure, or being formed from the same piece of material.

A distribution component may be detachable, and may be disposable, reusable, or recyclable. The dressing 110 and the container 115 are illustrative of distribution components. A fluid conductor is another illustrative example of a distribution component. A “fluid conductor,” in this context, may include a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from KCI of San Antonio, Tex.

A reduced-pressure supply, such as the reduced-pressure source 105, may be a reservoir of air at a reduced pressure, or may be a manual or electrically-powered device, such as a vacuum pump, a suction pump, a wall suction port available at many healthcare facilities, or a micro-pump, for example. “Negative pressure” or “reduced pressure” generally refers to a pressure less than a local ambient pressure, such as the ambient pressure in a local environment external to a sealed therapeutic environment. In many cases, the local ambient pressure may also be the atmospheric pressure at which a tissue site is located. Further, the pressure may be less than a hydrostatic pressure associated with tissue at the tissue site. Unless otherwise indicated, values of pressure stated herein are gauge pressures. References to increases in reduced pressure may refer to a decrease in absolute pressure, while decreases in reduced pressure may refer to an increase in absolute pressure. While the amount and nature of reduced pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with reduced-pressure therapy.

A controller, such as the controller 120, may be a microprocessor or computer programmed to operate one or more components of the therapy system 100, such as the reduced-pressure source 105. In some embodiments, for example, the controller 120 may be a microcontroller, which may include an integrated circuit containing a processor core and a memory programmed to directly or indirectly control one or more operating parameters of the therapy system 100. Operating parameters may include the power applied to the reduced-pressure source 105, the pressure generated by the reduced-pressure source 105, or the pressure distributed to the tissue interface 135, for example. The controller 120 may also be configured to receive one or more input signals, such as a feedback signal, and programmed to modify one or more operating parameters based on the input signals.

Sensors, such as the first sensor 125 and the second sensor 130, may be any apparatus operable to detect or measure a physical phenomenon or property, and generally provide a signal indicative of the phenomenon or property that is detected or measured. For example, the first sensor 125 and the second sensor 130 may be configured to measure one or more operating parameters of the therapy system 100. In some embodiments, the first sensor 125 may be a transducer configured to measure pressure in a pneumatic pathway and convert the measurement to a signal indicative of the pressure measured. In some embodiments, for example, the first sensor 125 may be a piezoresistive strain gauge. The second sensor 130 may optionally measure operating parameters of the reduced-pressure source 105, such as the voltage or current, in some embodiments. Signals from the first sensor 125 and the second sensor 130 may be suitable as an input signal to the controller 120, but some signal conditioning may be appropriate in some embodiments. For example, the signal may need to be filtered or amplified before it can be processed by the controller 120. Typically, the signal is an electrical signal, but may be represented in other forms, such as an optical signal.

The tissue interface 135 can be adapted to partially or fully contact a tissue site. The tissue interface 135 may take many forms, and may have many sizes, shapes, or thicknesses depending on a variety of factors, such as the type of treatment being implemented or the nature and size of a tissue site. For example, the size and shape of the tissue interface 135 may be adapted to the contours of deep and irregular shaped tissue sites. Moreover, any or all of the surfaces of the tissue interface 135 may have projections or an uneven, course, or jagged profile that can induce strains and stresses on a tissue site, which can promote granulation at the tissue site.

In some embodiments, the tissue interface 135 may be a manifold or may include a manifold and additional layers, such as a tissue contact layer, depending on the desired treatment. A “manifold” in this context may include any substance or structure providing a plurality of pathways adapted to collect or distribute fluid relative to a tissue. For example, a manifold may be adapted to receive reduced pressure from a source and distribute reduced pressure through multiple apertures to or from a tissue site, which may have the effect of collecting fluid from a tissue site and drawing the fluid toward the source. In some embodiments, the fluid path may be reversed or a secondary fluid path may be provided to facilitate delivering or moving fluid relative to a tissue site.

In some illustrative embodiments, the pathways of a manifold may be interconnected to improve distribution or collection of fluids at a tissue site. In some illustrative embodiments, a manifold may be a porous foam material having interconnected cells or pores. For example, open-cell foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively include projections that form interconnected fluid pathways. For example, a manifold may be molded to provide surface projections that define interconnected fluid pathways.

The average pore size of foam may vary according to needs of a prescribed therapy. For example, in some embodiments, the tissue interface 135 may be foam having pore sizes in a range of 400-600 microns. The tensile strength of the tissue interface 135 may also vary according to needs of a prescribed therapy. For example, the tensile strength of foam may be increased for instillation of topical treatment solutions. In some examples, the tissue interface 135 may be reticulated polyurethane foam such as found in GRANUFOAM™ dressing or V.A.C. VERAFLO™ dressing, both available from KCI of San Antonio, Tex.

The tissue interface 135 may be either hydrophobic or hydrophilic. In an example in which the tissue interface 135 may be hydrophilic, the tissue interface 135 may also wick fluid away from a tissue site, while continuing to distribute negative pressure to the tissue site. The wicking properties of the tissue interface 135 may draw fluid away from a tissue site by capillary flow or other wicking mechanisms. An example of hydrophilic foam is a polyvinyl alcohol, open-cell foam such as V.A.C. WHITEFOAM™ dressing available from KCI of San Antonio, Tex. Other hydrophilic foams may include those made from polyether. Other foams that may exhibit hydrophilic characteristics include hydrophobic foams that have been treated or coated to provide hydrophilicity.

The tissue interface 135 may further promote granulation at a tissue site when pressure within the sealed therapeutic environment is reduced. For example, any or all of the surfaces of the tissue interface 135 may have an uneven, coarse, or jagged profile that can induce microstrain and stress at a tissue site if negative pressure is applied through the tissue interface 135.

In some embodiments, the tissue interface 135 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 135 may further serve as a scaffold for new cell-growth, or a scaffold material may be used in conjunction with the tissue interface 135 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 140 may provide a bacterial barrier and protection from physical trauma. The cover 140 may also be constructed from a material that can reduce evaporative losses and provide a fluid seal between two components or two environments, such as between a therapeutic environment and a local external environment. For example, the cover 140 may comprise or consist essentially of an elastomeric film or membrane that can provide a seal adequate to maintain a reduced pressure at a tissue site for a given reduced-pressure source. In some example embodiments, the cover 140 may be a polymer drape, such as a polyurethane film, that is permeable to water vapor but impermeable to liquid. The cover 140 may have a high moisture-vapor transmission rate (MVTR) in some applications. For example, the MVTR may be at least 250 g/m\hat{ }2 per twenty-four hours in some embodiments (based on ASTM E96/E96M for upright cup measurement). Such drapes typically have a thickness in the range of 25-50 microns. For permeable materials, the permeability generally should be low enough that a desired negative pressure may be maintained.

An attachment device may be used to attach the cover 140 to an attachment surface, such as undamaged epidermis, a gasket, or another cover. The attachment device may take many forms. For example, an attachment device may be a medically-acceptable, pressure-sensitive adhesive configured to bond the cover 140 to epidermis around a tissue site. In some embodiments, for example, some or all of the cover 140 may be coated with an adhesive, such as an acrylic adhesive, which may have a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

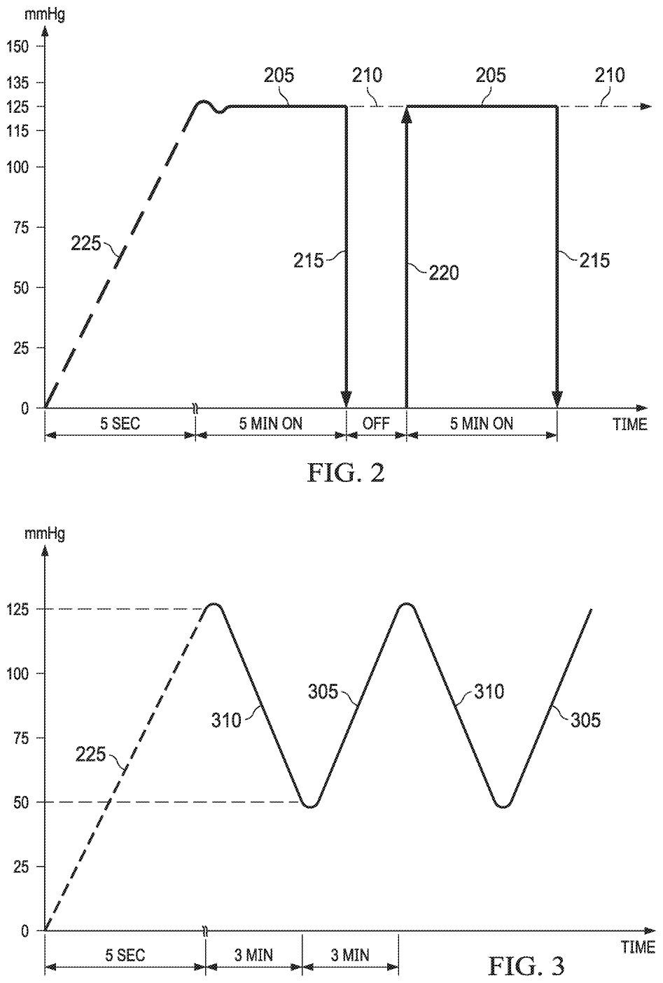

FIG. 2 is a graph illustrating additional details of an example control mode that may be associated with some embodiments of the controller 120. In some embodiments, the controller 120 may have a continuous pressure mode, in which the reduced-pressure source 105 is operated to provide a constant target reduced pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time, and the y-axis represents reduced pressure generated by the reduced-pressure source 105 over time. In the example of FIG. 2, the controller 120 can operate the reduced-pressure source 105 to cycle between a target pressure and atmospheric pressure. For example, the target pressure may be set at a value of 125 mmHg, as indicated by line 205, for a specified period of time (e.g., 5 min), followed by a specified period of time (e.g., 2 min) of deactivation, as indicated by the gap between the solid lines 215 and 220.

The cycle can be repeated by activating the reduced-pressure source 105, as indicated by line 220, which can form a square wave pattern between the target pressure and atmospheric pressure.

In some example embodiments, the increase in reduced-pressure from ambient pressure to the target pressure may not be instantaneous. For example, the reduced-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time as indicated by the solid line 220 may be a value substantially equal to the initial rise time as indicated by the dashed line 225.

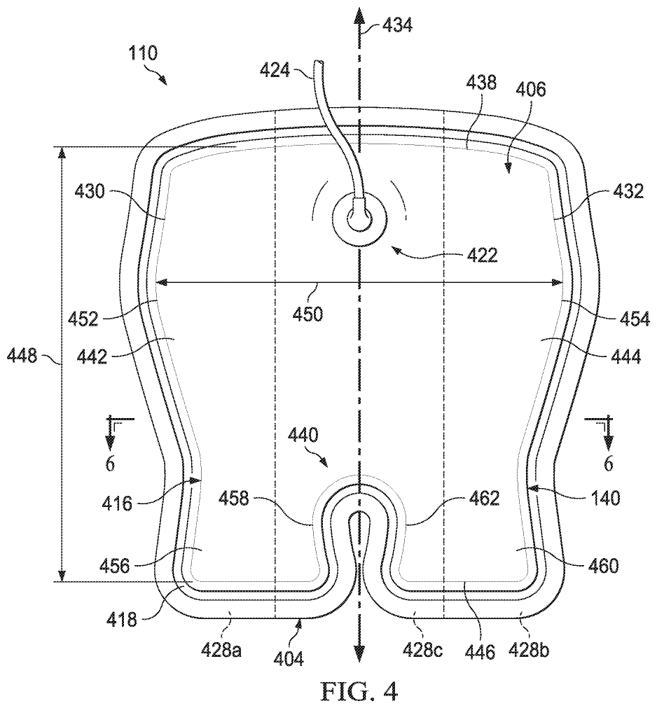

FIG. 3 is a graph illustrating additional details that may be associated with another example pressure control mode in some embodiments of the therapy system 100. In FIG. 3, the x-axis represents time and the y-axis represents negative pressure generated by the reduced-pressure source 105. The target pressure in the example of FIG. 3 can vary with time in a dynamic pressure mode. For example, the target pressure may vary in the form of a triangular waveform, varying between a minimum and maximum reduced pressure of 50-125 mmHg with a rise time 305 set at a rate of +25 mmHg/min. and a descent time 310 set at −25 mmHg/min, respectively. In other embodiments of the therapy system 100, the triangular waveform may vary between reduced pressure of 25-125 mmHg with a rise time 305 set at a rate of +30 mmHg/min and a descent time 310 set at −30 mmHg/min.

In some embodiments, the controller 120 may control or determine a variable target pressure in a dynamic pressure mode, and the variable target pressure may vary between a maximum and minimum pressure value that may be set as an input prescribed by an operator as the range of desired reduced pressure. The variable target pressure may also be processed and controlled by the controller 120, which can vary the target pressure according to a predetermined waveform, such as a triangular waveform, a sine waveform, or a saw-tooth waveform. In some embodiments, the waveform may be set by an operator as the predetermined or time-varying reduced pressure desired for therapy.

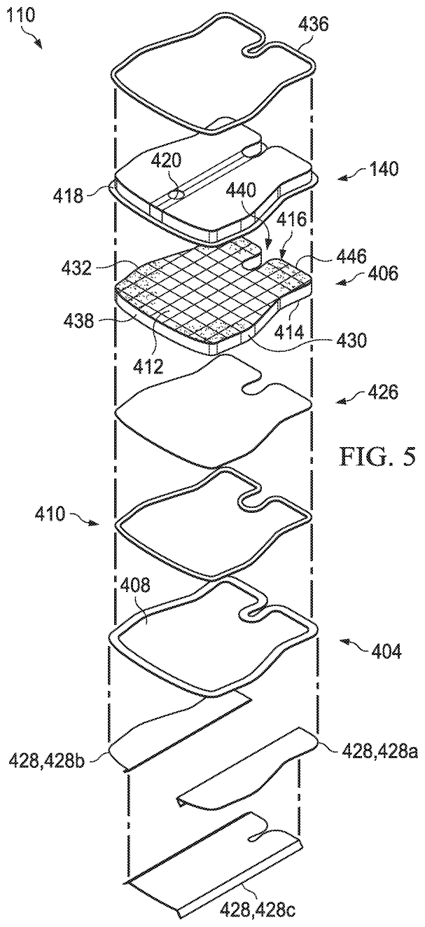

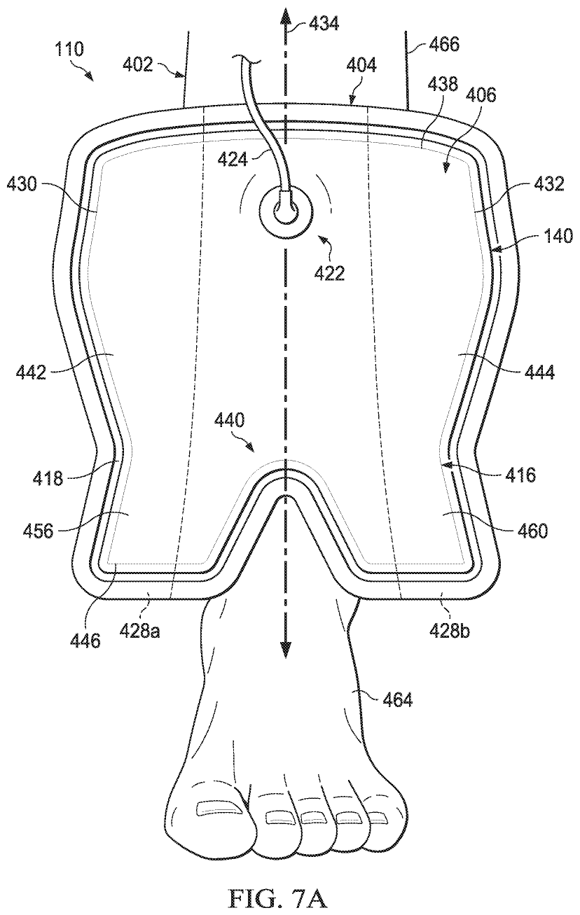

Referring to FIGS. 4-8, the dressing 110 may include features that can treat a tissue site at an extremity 402 of a patient, such as a leg, arm, ankle, wrist, or parts thereof, and an area of tissue around the tissue site and the extremity 402. For example, the tissue site may be an incision or other treatment target on one or both sides of a leg or ankle on a patient. The dressing 110 may be configured to treat not only the incision or treatment target, but also, an area of tissue around the incision or treatment target, the leg, and the ankle as desired.

Referring more specifically to FIGS. 4-6, in some examples, the dressing 110 may include an attachment device 404, a manifold 406, and the cover 140. Some examples of the attachment device 404 and other components may include a treatment aperture 408, and the manifold 406 may be configured to be at least partially exposed to an area of tissue around the extremity 402 through the treatment aperture 408. Further, in some examples, the dressing 110 may optionally include an adhesive ring 410 that may be configured to bond a peripheral portion of the manifold 406 to a portion of the attachment device 404. In some examples, the adhesive ring 410 may be formed as part of the attachment device 404, or the adhesive ring 410 may be omitted with the attachment device 404 instead being coupled to the manifold 406 with another medically acceptable coupling apparatus. In some examples, the cover 140, the manifold 406, the optional adhesive ring 410, and the attachment device 404 may have similar shapes. The attachment device 404 may be slightly larger than the manifold 406 to permit coupling of the attachment device 404 to the cover 140 around the manifold 406. In some examples, an adhesive may be disposed on a portion of the manifold 406 exposed through the treatment aperture 408. In some embodiments, the adhesive may be pattern-coated, and may cover up to 50% of the exposed portion or surface of the manifold 406.

The cover 140, the manifold 406, the attachment device 404, or various combinations may be assembled before application or at a treatment site. In some embodiments, the dressing 110 may be provided as a single unit.

The manifold 406 may include a first surface 412 and an opposing second surface 414. In some examples, at least a portion of the second surface 414 of the manifold 406 may be configured to face the area of tissue around the extremity 402 through the treatment aperture 408. In some examples, the attachment device 404 may be positioned on or at a portion of the second surface 414 of the manifold 406. In some examples, the manifold 406 may include or be formed of a porous material, such as foam.

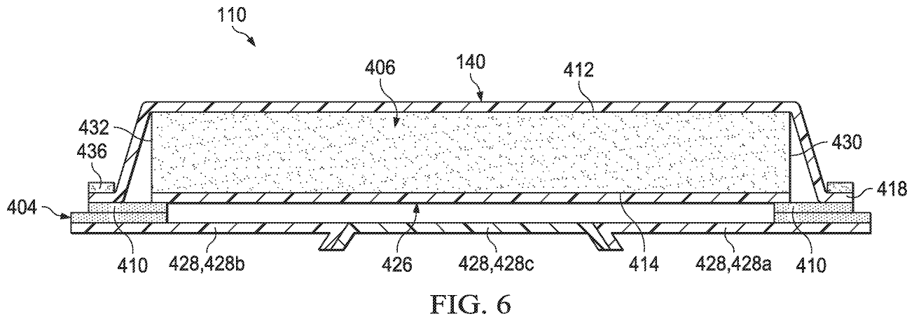

In some examples, the attachment device 404 may be configured to create a sealed space between the cover 140 and an area of tissue around the extremity 402, and the manifold 406 may be configured to be positioned in the sealed space. For example, the attachment device 404 may be positioned around an edge 416 of the manifold 406 and configured to surround the area of tissue around the extremity 402. The cover 140 may be disposed over the manifold 406 and coupled to the attachment device 404 around the manifold 406. For example, the cover 140 may be coupled to a portion of the attachment device 404 extending outward from the edge 416 of the manifold 406. Further, the cover 140 may be larger than the manifold 406, as illustrated in the example of FIG. 5, and may have a perimeter or a flange 418 configured to be attached to the attachment device 404. Assembled, the cover 140 may be disposed over the first surface 412 of the manifold 406, and the flange 418 may be attached to the attachment device 404 around the manifold 406. For example, an adhesive may be used to adhere the flange 418 to the attachment device 404, or the flange 418 may be, without limitation, welded, stitched, or stapled to the attachment device 404. The cover 140 may also include an aperture 420 configured to allow fluid communication between the manifold 404 and a dressing interface 422 and/or a fluid conductor 424 as described herein.

The attachment device 404 may take many forms. In some examples, the attachment device 404 may include or be formed of a film or membrane that can provide a seal in a therapeutic reduced-pressure environment. In some example embodiments, the attachment device 404 may be a polymer film, such as a polyurethane film, that is permeable to water vapor but impermeable to liquid. The attachment device 404 may have a thickness in the range of 25-50 microns. For permeable materials, the permeability may be low enough that a desired reduced pressure may be maintained. The attachment device 404 may also include a medically-acceptable adhesive, such as a pressure-sensitive adhesive. In examples, the attachment device 404 may be a polymer film coated with an adhesive, such as an acrylic adhesive, which may have a coating weight between 25-65 grams per square meter (g.s.m.). Thicker adhesives, or combinations of adhesives, may be applied in some examples to improve the seal and reduce leaks.

In some examples, the attachment device 404 may include or be formed of a hydrocolloid. In some examples, the attachment device 404 may be configured or referred to as a sealing ring or a gasket member. In other examples, the dressing 110 may include a gasket member (not shown) in addition to the attachment device 404. In such an example, the gasket member may be a peripheral member, such as a hydrocolloid ring, and at least a portion of the attachment device 404 may be positioned between the manifold 406 and the gasket member on or at a surface of the manifold 406, such as the second surface 414, configured to face the area of tissue around the extremity 402. In some examples, the gasket member may have a similar or analogous shape as the adhesive ring 410, but the gasket member may be positioned on a surface of the attachment device 404 configured to face the extremity 402 such that the gasket member is configured to be positioned between the extremity 402 and the attachment device 404.

In some examples, the dressing 110 may further include a tissue contact layer 426, which may be coupled to a surface of the manifold 406, such as the second surface 414, configured to be exposed to the area of tissue around the extremity 402. The tissue contact layer 426 may be configured to be positioned in direct contact with the area of tissue around the extremity 402. The tissue contact layer 426 may include or be formed of a material that substantially reduces or eliminates skin irritation while allowing fluid transfer through the tissue contact layer 426. In some examples, the tissue contact layer 426 may include or be formed of one or more of the following materials, without limitation: a woven material, a non-woven material, a polyester knit material, and a fenestrated film.

In some examples, the attachment device 404 or adhesive on a surface of the dressing 110 configured to face the area of tissue around the extremity 402 may be covered by one or more release liners 428 prior to applying the dressing 110 at the tissue site. For example, as shown in FIG. 5, the dressing 110 may include a first release liner 428a, a second release liner 428b, and a third release liner 428c. The first release liner 428a may be positioned proximate to a first side 430 of the manifold 406 or the dressing 110, the second release liner 428b may be positioned proximate to a second side 432 of the manifold 406 or the dressing 110, and the third release liner 428c may be positioned proximate to a fold axis 434 of the manifold 406 or the dressing 110. The third release liner 428c may be positioned between the first release liner 428a and the second release liner 428b. In some examples, the third release liner 428c may be configured to be removed to expose an adhesive or portion of the attachment device 404 proximate to the fold axis 434 prior to removal of the first release liner 428a and the second release liner 428b. Such a configuration may permit the fold axis 434 of the dressing 110 to be initially positioned or aligned at a tissue site, such as the extremity 402, while the first release liner 428a and the second release liner 428b protect other portions of the adhesive or the attachment device 404. For example, a portion of the third release liner 428c may cover or be positioned over a portion of the first release liner 428a and/or the second release liner 428b such that the third release liner 428c may be removed prior to removal of the first release liner 428a and the second release liner 428b. In some examples, the dressing 110 may have two release liners, each of which may have perforations or slits (not shown) configured to allow the release liners to be separated into smaller pieces for removal. Additionally, some embodiments may also have one or more casting sheet liners 436.

Additionally or alternatively, the first release liner 428a, the second release liner 428b, and the third release liner 428c may provide stiffness to the attachment device 404 to facilitate handling and application. Additionally or alternatively, the casting sheet liners 436 may cover the flange 418 to provide stiffness to the cover 140 for handling and application.

In some examples, the dressing 110 may include the dressing interface 422, which may be fluidly coupled to the manifold 406 through the aperture 420 in the cover 140. The dressing interface 422 may be coupled toward a second end 438 of the manifold 406, and may be configured to be coupled to the reduced-pressure source 105 through, for example, the fluid conductor 424, conduit, or tube coupled in fluid communication between the dressing interface 422 and the reduced pressure source 105.

In some examples, the manifold 406 may include a concave recess 440, the fold axis 434, a first portion or conformable area 442, and a second portion or conformable area 444. The concave recess 440 may be positioned at a first end 446 of the manifold 406 and may extend into the edge 416 of the manifold 406 at the first end 446. In some examples, the concave recess 440 may extend into the edge 416 of the manifold 406 between about 15 percent to about 30 percent of a length 448 of the manifold 406 between the first end 446 and the second end 438. In some examples, the concave recess 440 may form or be configured in an arch or a V-shape.

The fold axis 434 may bisect the concave recess 440 and extend from the first end 446 of the manifold 406 to the second end 438 of the manifold 406 along the length 448 of the manifold 406. The first end 446 of the manifold 406 may be positioned opposite from the second end 438 of the manifold 406 along the length 448 of the manifold 406. The first conformable area 442 may extend orthogonal to the fold axis 434 along a width 450 of the manifold 406 and toward the first side 430 of the manifold 406. The second conformable area 444 may extend orthogonal to the fold axis 434 along the width 450 of the manifold 406 and toward the second side 432 of the manifold 406 opposite to the first side 430.

In some examples, at least a portion of the width 450 of the manifold 406 may increase from the first end 446 of the manifold 406 to the second end 438 of the manifold 406. For example, the first conformable area 442 may include a first flared portion 452 and the second conformable area 444 may include a second flared portion 454. The width 450 of the manifold 406 may be greatest from the first flared portion 452 to the second flared portion 454. In some embodiments, the width 450 of the manifold 406 between the first flared portion 452 and the second flared portion 454 may be between about 26 centimeters to about 30 centimeters. Further, the length 448 of the manifold 406 between the first end 446 and the second end 438 may be between about 27 centimeters to about 30 centimeters.

In some examples, the first flared portion 452 and the second flared portion 454 may be positioned closer to the second end 438 of the manifold 406 than the first end 446 of the manifold 406. In some examples, the first flared portion 452 may be positioned at the edge 416 of the manifold 406 on the first side 430 of the manifold 406, and the second flared portion 454 may be positioned at the edge 416 of the manifold 406 on the second side 432 of the manifold 406.

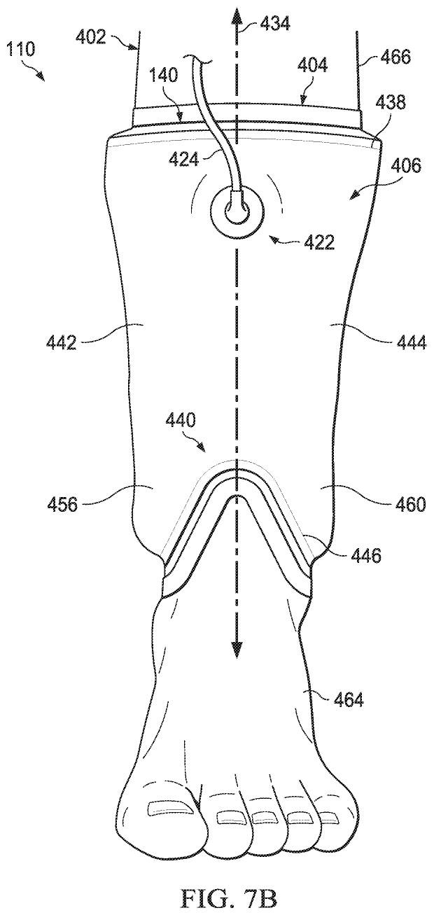

In some examples, the dressing 110 may include a first flap 456 in the first conformable area 442 partially defined by a first bisected portion 458 of the concave recess 440, and a second flap 460 in the second conformable area 444 partially defined by a second bisected portion 462 of the concave recess 440. The concave recess 440 may be positioned between the first flap 456 and the second flap 460. In some examples, the first flap 456 and the second flap 460 may be configured to be positioned on opposing sides of an ankle as shown in FIGS. 7A-7B. Further, in some examples, the first conformable area 442 may be symmetrical to the second conformable area 444 across the fold axis 434.

The thickness of the manifold 406, for example, between the first surface 412 and the second surface 414, may vary according to prescribed therapy. In some examples, the manifold 406 or a portion of the manifold 406, may include felted, open-cell foam configured to increase rigidity. Additionally or alternatively, the manifold 406 may include foam segments having different density.

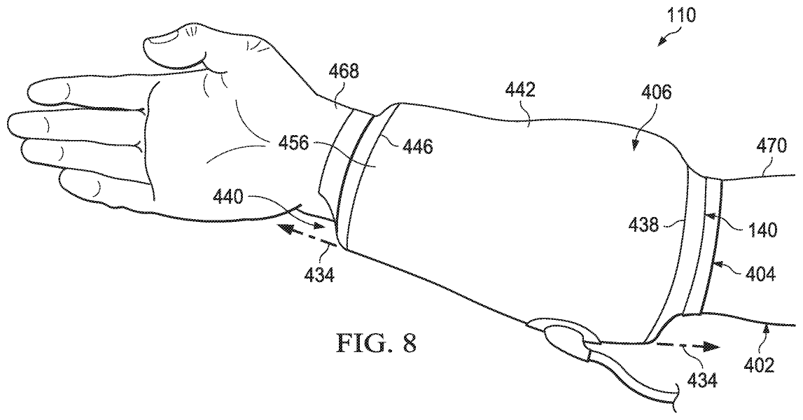

Referring to FIGS. 7A-7B, in some examples, the concave recess 440 of the dressing 110 may be sized and configured to receive a dorsal portion of a foot 464. In such an example, the fold axis 434 may be configured to extend lengthwise along a shin of a leg 466, and the first portion or conformable area 442 and the second portion or conformable area 444 may be configured to wrap around an ankle and a calf of the leg 466. In other examples, the concave recess 440 of the dressing 110 may be sized and configured to receive a portion of a wrist 468 on an arm 470 of a patient as shown in FIG. 8. In such an example, the fold axis 434 may be configured to extend lengthwise along arm 470, and the first conformable area 442 and the second conformable area 444 may be configured to wrap around the wrist 468 and the arm 470.

Some examples of a method for treating an area around an extremity 402 with reduced pressure may also be illustrated with reference to FIGS. 7A-B. In some examples, such a method may include applying the dressing 110 to the extremity 402 such that the fold axis 434 extends lengthwise along the extremity 402. Further, the method may include wrapping the first conformable area 442 or portion circumferentially around the extremity 402 in a first direction, and wrapping the second conformable area 444 or portion circumferentially around the extremity 402 in a second direction opposite to the first direction. Further, the method may include fluidly coupling the reduced-pressure source 105 to the manifold 406 and delivering reduced pressure from the reduced-pressure source 105 to the manifold 406. In some examples, the method may include applying the fold axis 434 lengthwise along a shin of the leg 466, and positioning a dorsal portion of the foot 464 on the leg 466 in the concave recess 440. Further, in some examples, the method may include positioning the first flap 456 and the second flap 460 on opposing sides of an ankle.

Further, in some examples, the dressing 110 may further include an attachment device or an adhesive on a surface of the dressing 110 configured to face the area of tissue around the extremity 402 and the plurality of release liners 428 covering the attachment device or the adhesive prior to applying the dressing 110. The method may further include removing one or more of the plurality of release liners 428, such as the third release liner 428c, covering the attachment device or the adhesive in a location proximate to the fold axis 434 before applying the fold axis 434 lengthwise along the extremity 402 and before removing another of the release liners 428 or wrapping the first conformable area 442 or the second conformable area 444.

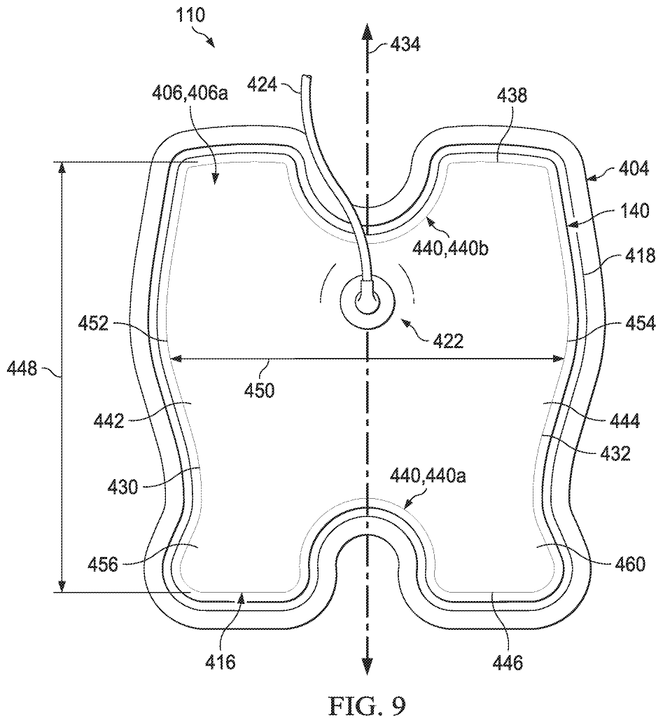

Referring to FIGS. 9-11, the dressing 110 and the manifold 406 may include additional shapes as shown to facilitate or enhance the ability of the dressing 110 and the manifold 406 to conform to various types and sizes of tissue sites and extremities as desired. Referring to FIG. 9, the manifold 406 may be a manifold 406a, wherein like reference numerals refer to like features or elements described in association with other example embodiments. In some examples, the concave recess 440 may be a first concave recess 440a and the manifold 406a may additionally include a second concave recess 440b extending into the edge 416 of the manifold 406a at the second end 438 of the manifold 406a. In some examples, the second concave recess 440b may be larger than the first concave recess 440a, which can enhance the ability of the manifold 406 to fit a large size range of patients.

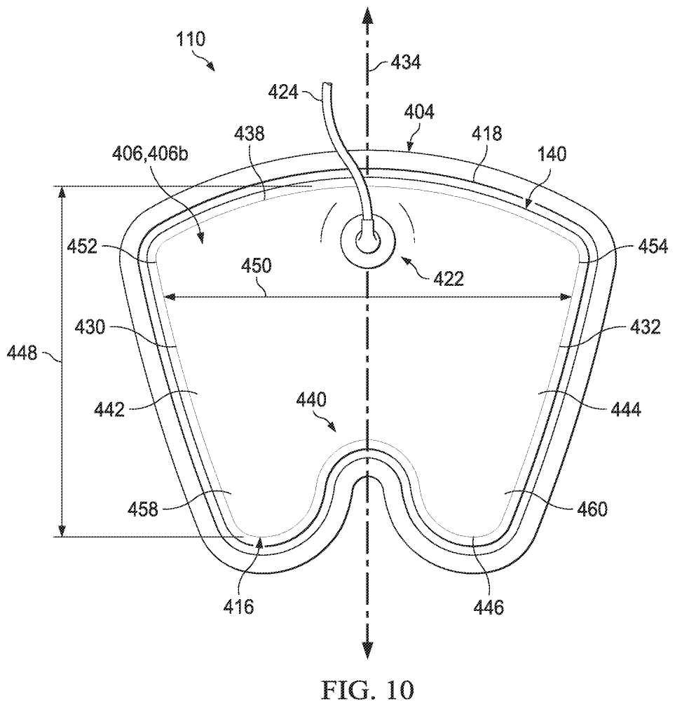

Referring to FIG. 10, in some examples, the manifold 406 may be a manifold 406b, wherein like reference numerals refer to like features or elements described in association with other example embodiments. The length 448 of the manifold 406b of the example of FIG. 10 may be between about 8 inches to about 10 inches. Further, the width 450 of the example manifold 406b proximate to the first end 446 and between the first side 430 and the second side 432 may be between about 6 inches to about 8 inches. Further, the width 450 of the example manifold 406b may increase toward, at, or proximate to the second end 438 to a value between about 11 inches to about 13 inches. The first flared portion 452 and the second flared portion 454 of the example manifold 406b may be positioned on the second end 438 of the manifold 406b, and the width 450 of the manifold 406b may be greatest between the first flared portion 452 and the second flared portion 454.

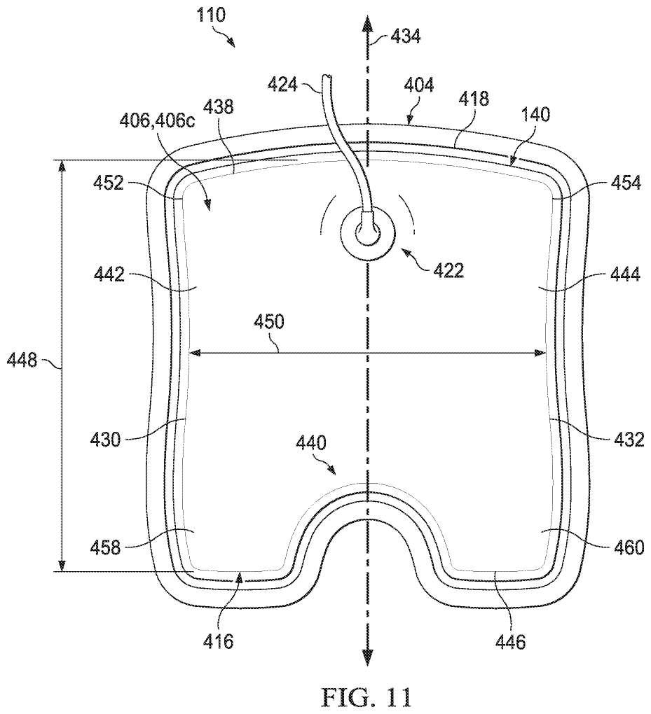

Referring to FIG. 11, in some examples, the manifold 406 may be a manifold 406c, wherein like reference numerals refer to like features or elements described in association with other example embodiments. The length 448 of the manifold 406c of the example of FIG. 11 may be between about 8 inches to about 10 inches. Further, the width 450 of the example manifold 406c proximate to the first end 446 and between the first side 430 and the second side 432 may be between about 6 inches to about 8 inches. Further, the width 450 of the example manifold 406c may increase toward, at, or proximate to the second end 438 to a value between about 7 inches to about 9 inches, which is smaller and less tapered than the example of FIG. 10. Further, the first flared portion 452 and the second flared portion 454 of the example manifold 406c may be positioned on the second end 438 of the manifold 406c, and the width 450 of the manifold 406c may be greatest between the first flared portion 452 and the second flared portion 454.

In operation, the reduced-pressure source 105 can reduce pressure in the sealed therapeutic environment. Reduced pressure applied to the tissue site through the manifold 406 in the sealed therapeutic environment can induce macro-strain and micro-strain in the tissue site, as well as remove exudates and other fluids from the tissue site, which can be collected in the container 115.

In general, exudates and other fluids flow toward lower pressure along a fluid path. Thus, the term “downstream” may refer to a location in a fluid path relatively closer to a source of reduced pressure or further away from a source of positive pressure. Conversely, the term “upstream” may refer to a location further away from a source of reduced pressure or closer to a source of positive pressure.

In some example embodiments, the controller 120 may receive and process data from one or more sensors, such as the first sensor 125. The controller 120 may also control the operation of one or more components of the therapy system 100 to manage the pressure delivered to the tissue interface 135, such as the manifold 406 and associated components. In some embodiments, the controller 120 may include an input for receiving a desired target pressure, and may be programmed for processing data relating to the setting and inputting of the target pressure to be applied to the tissue interface 135. In some example embodiments, the target pressure may be a fixed pressure value set by an operator as the target reduced pressure desired for therapy at a tissue site and then provided as input to the controller 120. The target pressure may vary from tissue site to tissue site based on the type of tissue forming a tissue site, the type of injury or wound (if any), the medical condition of the patient, and the preference of the attending physician. After selecting a desired target pressure, the controller 120 can operate the reduced-pressure source 105 in one or more control modes based on the target pressure, and may receive feedback from one or more sensors to maintain the target pressure at the tissue interface 135. In some embodiments, the manifold 406 may have distinct pressure zones, and different target pressures and control modes may be applied to different pressure zones.

The systems, apparatuses, and methods described herein may provide significant advantages. For example, in addition to the benefits of increased development of granulation tissue and reduced healing times, the system 100 can also reduce edema and bruising in a broader area of tissue surrounding or adjacent to a tissue site or treatment target, such as an incision. The dressing 110, for example, can reduce stress on an incision and maximize the treatment coverage area of patient extremities. The dressing 110 can also be beneficial for managing edema and bruising of tissue sites without an incision or open wound, such as a sprain.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 120 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating an area around an extremity with reduced pressure, comprising:
an attachment device including a treatment aperture;
a manifold configured to be at least partially exposed to the area around the extremity through the treatment aperture and including:
a concave recess extending into an edge of the manifold at a first end,
a fold axis bisecting the concave recess and extending from the first end to a second end along a length of the manifold,
a first conformable area extending orthogonal to the fold axis along a width of the manifold and toward a first side of the manifold, and
a second conformable area extending orthogonal to the fold axis along the width of the manifold and toward a second side of the manifold opposite to the first side, wherein at least a portion of the width of the manifold increases from the first end to the second end; and
a cover configured to be disposed over the manifold and coupled to the attachment device around the manifold.

2. The dressing of claim 1, wherein the concave recess extends into the edge of the manifold between about 15 percent to about 30 percent of the length of the manifold between the first end and the second end.

3. (canceled)

4. (canceled)

5. The dressing of claim 1, wherein the concave recess is a first concave recess and wherein the manifold further comprises a second concave recess extending into the edge of the manifold at the second end, and wherein the second concave recess is larger than the first concave recess.

6. The dressing of claim 1, wherein the first conformable area includes a first flared portion and the second conformable area includes a second flared portion, and wherein the width of the manifold is greatest from the first flared portion to the second flared portion.

7. The dressing of claim 6, wherein the first flared portion and the second flared portion are positioned closer to the second end of the manifold than the first end.

8. The dressing of claim 6, wherein the first flared portion is positioned at the edge of the manifold on the first side and the second flared portion is positioned at the edge of the manifold on the second side.

9. The dressing of claim 1, further comprising a first flap in the first conformable area partially defined by a first bisected portion of the concave recess, and a second flap in the second conformable area partially defined by a second bisected portion of the concave recess, wherein the concave recess is positioned between the first flap and the second flap, and wherein the first flap and the second flap are configured to be positioned on opposing sides of an ankle.

10. (canceled)

11. The dressing of claim 1, wherein the first conformable area is symmetrical to the second conformable area across the fold axis.

12. The dressing of claim 1, wherein the attachment device is configured to create a sealed space between the cover and the area around the extremity, wherein the manifold is configured to be positioned in the sealed space, and wherein the manifold comprises porous foam.

13. (canceled)

14. The dressing of claim 1, wherein the attachment device comprises one or more of a film layer, an adhesive, a hydrocolloid, and a gasket member.

15. (canceled)

16. (canceled)

17. The dressing of claim 1, wherein the attachment device is positioned around the edge of the manifold and configured to surround the area around the extremity.

18. The dressing of claim 1, wherein the manifold includes a first surface and an opposing second surface, wherein at least of portion of the second surface of the manifold is configured to face the area around the extremity through the treatment aperture, and wherein the attachment device is positioned on a portion of the second surface of the manifold.

19. The dressing of claim 18, wherein the cover is configured to be coupled to a portion of the attachment device extending outward from the edge of the manifold.

20. (canceled)

21. The dressing of claim 1, further comprising a tissue contact layer coupled to a surface of the manifold configured to be exposed to the area around the extremity, wherein the tissue contact layer is configured to be positioned in direct contact with the area around the extremity, and wherein the tissue contact layer is selected from the group consisting of a woven material, a non-woven material, a polyester knit material, and a fenestrated film.

22. (canceled)

23. (canceled)

24. A method for treating an area around an extremity with reduced pressure, comprising:
applying the dressing of claim 1 so that the fold axis extends lengthwise along the extremity;
wrapping the first conformable area circumferentially around the extremity in a first direction;
wrapping the second conformable area circumferentially around the extremity in a second direction opposite to the first direction;
fluidly coupling a reduced-pressure source to the manifold; and
delivering reduced pressure from the reduced-pressure source to the manifold.

25. The method of claim 24, wherein the extremity is a leg, and wherein applying the dressing includes applying the fold axis lengthwise along a shin of the leg and positioning a dorsal portion of a foot on the leg in the concave recess.

26. The method of claim 24, wherein the dressing further comprises a first flap and a second flap, and wherein the method further comprises positioning the first flap and the second flap on opposing sides of an ankle.

27. The method of claim 24, wherein the dressing further comprises an adhesive on a surface of the dressing configured to face the area around the extremity and a plurality of release liners covering the adhesive prior to applying the dressing, and wherein the method further comprises removing one or more of the plurality of release liners covering the adhesive at the fold axis before applying the fold axis lengthwise along the extremity and before removing another of the release liners or wrapping the first conformable area or the second conformable area.

28. A manifold for use with a dressing for treating an area around an extremity with reduced pressure, the manifold comprising:
a concave recess extending into an edge of the manifold at a first end;
a fold axis bisecting the concave recess and extending from the first end to a second end along a length of the manifold;
a first conformable area extending orthogonal to the fold axis along a width of the manifold and toward a first side of the manifold; and
a second conformable area extending orthogonal to the fold axis along the width of the manifold and toward a second side of the manifold opposite to the first side;
wherein the first conformable area is symmetrical to the second conformable area across the fold axis.

29.-36. (canceled)

37. A manifold for use with a dressing for treating an area around an extremity with reduced pressure, the manifold comprising:
a concave recess at a first end of the manifold;
a fold axis bisecting the concave recess and extending from the first end to a second end of the manifold;
a first portion extending orthogonal to the fold axis toward a first side of the manifold; and
a second portion extending orthogonal to the fold axis toward a second side of the manifold opposite to the first side;
wherein the fold axis is configured to extend lengthwise along the extremity.

38. (canceled)

39. (canceled)
